(19) United States
(12) Patent Application Publication
(10) Pub. No.: US 2019/0373871 A1
(43) Pub. Date: Dec. 12, 2019

(54) METHOD FOR ASSAYING GENETIC VARIANTS

(71) Applicant: RADY CHILDREN'S HOSPITAL RESEARCH CENTER, San Diego, CA (US)

(72) Inventor: Matthew Bainbridge, San Diego, CA (US)

(21) Appl. No.: 16/438,010

(22) Filed: Jun. 11, 2019

Related U.S. Application Data
(60) Provisional application No. 62/684,039, filed on Jun. 12, 2018.

(51) Int. Cl. A01K 67/033; C12N 15/90; C40B 40/08; C12N 15/85; C40B 30/06

(52) U.S. Cl. CPC A01K 67/0336; C12N 15/90; C40B 40/08; C12N 15/85; A01K 2267/0393; C12N 2015/8536; A01K 2217/15; A01K 2227/703; C40B 30/06

(57) ABSTRACT

The present disclosure provides a method for genetic analysis of gene variants as well as a system for implementing such analysis.

### CROSS REFERENCE TO RELATED APPLICATION

This application claims the benefit of U.S. Provisional Patent Application Ser. No. 62/684,039, filed on Jun. 12, 2018. The entire contents of the foregoing is hereby incorporated by reference in its entirety.

### BACKGROUND OF THE INVENTION

### Field of the Invention

The invention relates generally to genetic analysis and more specifically to a method and system for assaying genes, such as seizure genes in an animal model.

### Background Information

The ability to identify which gene variants cause disease and which do not, and which of those will respond to therapy is a central issue in modern genetics. Being able to simultaneously test all variants in a gene and evaluate their pathogenicity as well as genotype-specific drug response is the holy grail of precision medicine.

High-throughput assays for evaluating the effects of all mutations often share the following commonalities: 1) they use high-throughput oligo-manufacturing to produce cDNAs from a gene that contain all possible amino acid substitutions; 2) they use some assay/selection to evaluate the effect of the mutation on gene function; and 3) they use high-throughput sequencing to measure the relative amount of a particular substitution before and after the selection process.

There exists a need for improved assay methods for identifying gene variants, as well as performing drug screening in a genotype-specific manner.

### SUMMARY OF THE INVENTION

The present invention provides a method and system for conducting genetic analysis. The invention provides for detection of pathogenic gene variants.

Accordingly, in embodiments, the invention provides a method for performing genetic analysis. In one embodiment, the method includes:

a) humanizing a worm by integrating a human ortholog into the worm genome;
b) transfecting a worm population with a library of oligonucleotides wherein the library of oligonucleotides contain genetic variants of the ortholog;
c) detecting the relative proportions of each genetic variant in the worm population;
d) conducting a chemotaxis assay using worms of the worm population; and
e) detecting worms that respond to a stimulus in the chemotaxis assay and worms that fail to respond to the stimulus and measuring the relative proportion of each ortholog variant in each group of worms.

The invention further provides a method for identifying a pathogenic genetic mutation in a gene using a human worm ortholog model. The method includes:

a) humanizing a worm by integrating a human ortholog into the worm genome;
b) transfecting a worm population with a library of oligonucleotides wherein the library of oligonucleotides contain genetic variants of the ortholog;
c) detecting the relative proportions of each genetic variant in the worm population;
d) conducting a chemotaxis assay using worms of the worm population; and
e) detecting worms that respond to a stimulus in the chemotaxis assay and worms that fail to respond to the stimulus and measuring the relative proportion of each ortholog variant in each group of worms, wherein worms that do not respond to the stimulus have a genetic variant of the ortholog that is pathogenic, thereby identifying a pathogenic genetic mutation.

In yet another embodiment, the invention provides a method for screening a test agent in a human worm ortholog model. The method includes:

a) humanizing a worm by integrating a human ortholog into the worm genome;
b) transfecting a worm population with a library of oligonucleotides wherein the library of oligonucleotides contain genetic variants of the ortholog;
c) detecting the relative proportions of each genetic variant in the worm population;
d) conducting a chemotaxis assay using worms of the worm population, wherein the assay is conducted in the presence and absence of a test compound; and
e) determining whether the test compound alters function of the ortholog by detecting an increase or decrease in chemotaxis activity by comparing chemotaxis activity in the presence and absence of the compound.

In embodiments, the library of oligonucleotides includes oligonucleotides having at least 50, 60, 70, 80, 90% or more of all possible amino acid substitutions, deletions and/or mutations of the human ortholog. In one embodiment, the library of oligonucleotides includes oligonucleotides having all possible amino acid substitutions, deletions and/or mutations of the human ortholog.

In embodiments, the ortholog is a gene associated with seizure, neuro-conduction, movement disorders (e.g. ataxia, dystonia), or muscle disorder in humans.

In embodiments, the method of the invention is used to identify genetic variants of a gene. In one embodiment, the method of the invention is used to identify every possible genetic variant of a gene.

In embodiments, the method of the invention is used to classify a genetic variant as pathogenic or non-pathogenic.

In embodiments, the method of the invention is used to perform drug screening in a mutation specific manner.

In another embodiment, the invention provides a system for performing the method of the invention. The system includes a controller having at least one processor and non-transitory memory. The controller is configured to perform one or more of the processes of the method as described herein.

### BRIEF DESCRIPTION OF THE DRAWINGS

FIG. 1 is a graph showing experimental data generated by the method of the disclosure in one embodiment.

### DETAILED DESCRIPTION OF THE INVENTION

The present invention is based on an innovative method for identifying and screening gene variants. The method may be used in a number of ways, including, for example, to identify every possible genetic variant of a gene, to classify a genetic variant as pathogenic or non-pathogenic, or to perform drug screening in a mutation specific manner.

Conventional assays are often hampered by the step of identifying an appropriate selection process that is effective to evaluate the effect of a mutation on gene function. These assays tend to be very gene specific and not widely applicable. The present disclosure proposes use of a C. elegans chemotaxis assay as a mechanism to select for variants and drugs that alter chemotaxis. It is known that many human disease genes can affect chemotaxis in worms, including seizure genes. This is beneficial because it represents a large class of genes that could be tested with an identical assay.

Before the present compositions and methods are described, it is to be understood that this invention is not limited to particular methods and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

As used herein, a “human ortholog” refers to a human gene that is orthologous to a worm gene, namely a Caenorhabditis elegans gene.

Methods

In one aspect the invention provides a method for conducting genetic analysis. In embodiments, the invention provides for detection and/or classification of gene variants.

Accordingly, in one embodiment, the invention provides a method for identifying a human worm ortholog, The method includes:

a) humanizing a worm by integrating a human ortholog into the worm genome;
b) transfecting a worm population with a library of oligonucleotides wherein the library of oligonucleotides contain genetic variants of the ortholog;
c) detecting the relative proportions of each genetic variant in the worm population;
d) conducting a chemotaxis assay using worms of the worm population; and
e) detecting worms that respond to a stimulus in the chemotaxis assay and worms that fail to respond to the stimulus and measuring the relative proportion of each ortholog variant in each group of worms.

The invention further provides a method for identifying a pathogenic genetic mutation in a gene using a human worm ortholog model. The method includes:

a) humanizing a worm by integrating a human ortholog into the worm genome;
b) transfecting a worm population with a library of oligonucleotides wherein the library of oligonucleotides contain genetic variants of the ortholog;
c) detecting the relative proportions of each genetic variant in the worm population;
d) conducting a chemotaxis assay using worms of the worm population; and
e) detecting worms that respond to a stimulus in the chemotaxis assay and worms that fail to respond to the stimulus and measuring the relative proportion of each ortholog variant in each group of worms, wherein worms that do not respond to the stimulus have a genetic variant of the ortholog that is pathogenic, thereby identifying a pathogenic genetic mutation.

In yet another embodiment, the invention provides a method for screening a test agent in a human worm ortholog model. The method includes:

a) humanizing a worm by integrating a human ortholog into the worm genome;
b) transfecting a worm population with a library of oligonucleotides wherein the library of oligonucleotides contain genetic variants of the ortholog;
c) detecting the relative proportions of each genetic variant in the worm population;
d) conducting a chemotaxis assay using worms of the worm population, wherein the assay is conducted in the presence and absence of a test compound; and
e) determining whether the test compound alters function of the ortholog by detecting an increase or decrease in chemotaxis activity by comparing chemotaxis activity in the presence and absence of the compound.

In embodiments, the worm population is Caenorhabditis elegans.

Accordingly, in an embodiment, the disclosure provides a method which includes:

The methodology will include the following:

a) identify a human gene that is orthologous (human ortholog) to a worm gene that, when dysregulated in the worm, causes a defect in chemotaxis, such as CACNB4;
b) humanize the worm, by introducing the human ortholog cDNA into the worm, and observing that it restores chemotaxis (i.e., gene rescue);
c) generate gene variant oligonucleotides of ortholog gene variants having all possible amino acid substitutions;
d) transfect a C. elegans population with the gene variant oligonucleotides;
e) high throughput sequence a subset of the transfected worm population to measure the relative proportions of each gene variant;
f) conduct a chemotaxis assay; and
g) high-throughput sequence the worms that responded to the stimulus in the chemotaxis assay (either by moving toward or away from it) and those that did not to measure the relative abundance of each variant in each group.

A chemotaxis assay may include testing the mobility of worms in response to an external stimuli. The stimuli may be chemical, light, temperature, pressure or other type of stimuli generally known in the art. A positive response to the stimuli is measured by movement of the worm either toward or away from the stimuli. A negative response is little or no movement of the worm towards or away from the stimuli.

A hallmark of the invention is that worms do not need to die in order to identify pathogenic or lethal gene mutations. The methodology of the disclosure allows identification and/or separation of pathogenic from non-pathogenic gene mutations by the level of motility/locomotion of the worms. For example, worm motility may be compared to a negative control as well as a worm having a known genetic variant of a gene that includes a pathogenic or lethal mutation. In embodiments, motility of a worm having a gene variant with a mutation that is unidentified as pathogenic or lethal that is substantially similar or equal to that of a worm having a known pathogenic or lethal mutation is indicative of the unidentified gene variant as having a pathogenic or lethal mutation. Similarly, motility of a worm having a gene variant with a mutation that is unidentified as pathogenic or lethal that is substantially similar or equal to that of a worm having a known non-pathogenic or lethal mutation is indicative of the unidentified gene variant as having a non-pathogenic or lethal mutation.

The chemotaxis assay may be utilized for screening efficacy of a drug candidate that can ameliorate or reverse effects of the genetic mutation. This may be performed by introducing a drug candidate (i.e., test agent) into the chemotaxis assay and determining whether the drug candidate alters the mobility of the worms in the presence of the test agent and stimuli as compared to mobility of the worms in the absence of the test agent and stimuli.

The method of the disclosure contemplates genetic sequencing. For example, sequencing may be utilized in performing steps (c) and (e) of the method.

Sequencing may be by any method known in the art. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent™ sequencing), nanopore sequencing, pyrosequencing (454), sequencing by synthesis, sequencing by ligation (SOLD′ sequencing), sequencing by electron microscopy, dideoxy sequencing reactions (Sanger method), massively parallel sequencing, polony sequencing, and DNA nanoball sequencing. In some embodiments, sequencing involves hybridizing a primer to the template to form a template/primer duplex, contacting the duplex with a polymerase enzyme in the presence of a detectably labeled nucleotides under conditions that permit the polymerase to add nucleotides to the primer in a template-dependent manner, detecting a signal from the incorporated labeled nucleotide, and sequentially repeating the contacting and detecting steps at least once, wherein sequential detection of incorporated labeled nucleotide determines the sequence of the nucleic acid. In some embodiments, the sequencing comprises obtaining paired end reads.

In some embodiments, sequencing of nucleic acid is performed using whole genome sequencing (WGS) or rapid WGS. In some embodiments, targeted sequencing is performed and may be either DNA or RNA sequencing. The targeted sequencing may be to a subset of the whole genome. In some embodiments the targeted sequencing is to introns, exons, non-coding sequences or a combination thereof. In other embodiments, targeted whole exome sequencing (WES) of the DNA from the sample is performed. The DNA is sequenced using a next generation sequencing platform (NGS), which is massively parallel sequencing. NGS technologies provide high throughput sequence information, and provide digital quantitative information, in that each sequence read that aligns to the sequence of interest is countable. In certain embodiments, clonally amplified DNA templates or single DNA molecules are sequenced in a massively parallel fashion within a flow cell (e.g., as described in WO 2014/015084). In addition to high-throughput sequence information, NGS provides quantitative information, in that each sequence read is countable and represents an individual clonal DNA template or a single DNA molecule. The sequencing technologies of NGS include pyrosequencing, sequencing-by-synthesis with reversible dye terminators, sequencing by oligonucleotide probe ligation and ion semiconductor sequencing. DNA from individual samples can be sequenced individually (i.e., singleplex sequencing) or DNA from multiple samples can be pooled and sequenced as indexed genomic molecules (i.e., multiplex sequencing) on a single sequencing run, to generate up to several hundred million reads of DNA sequences. Commercially available platforms include, e.g., platforms for sequencing-by-synthesis, ion semiconductor sequencing, pyrosequencing, reversible dye terminator sequencing, sequencing by ligation, single-molecule sequencing, sequencing by hybridization, and nanopore sequencing. In embodiments, the methodology of the disclosure utilizes systems such as those provided by Illumina, Inc, (NovaSeq, NextSeq, HiSeg™ X10, HiSeg™ 1000, HiSeg™ 2000, HiSeg™ 2500, Genome Analyzers™, MiSeg™ systems), Applied Biosystems Life Technologies (SOLiD™ System, Ion PGM™ Sequencer, ion Proton™ Sequencer). Nucleic acid analysis can also be carried out by systems provided by BGI or BGI Americas or affiliates. Nucleic acid analysis can also be carried out by systems provided by Oxford Nanopore Technologies (GridiON™, MiniON™) or Pacific Biosciences (Pacbio™ RS II). Importantly, in embodiments, sequencing may be performed using any of the methods described herein.

As used herein, the term “mutation” herein refers to a change introduced into a reference sequence, including, but not limited to, substitutions, insertions, deletions (including truncations) relative to the reference sequence. Mutations can involve large sections of DNA (e.g., copy number variation). Mutations can involve small sections of DNA. Examples of mutations involving small sections of DNA include, e.g., point mutations or single nucleotide polymorphisms (SNPs), multiple nucleotide polymorphisms, insertions (e.g., insertion of one or more nucleotides at a locus but less than the entire locus), multiple nucleotide changes, deletions (e.g., deletion of one or more nucleotides at a locus), and inversions (e.g., reversal of a sequence of one or more nucleotides). The consequences of a mutation include, but are not limited to, the creation of a new character, property, function, phenotype or trait not found in the protein encoded by the reference sequence.

As used herein, a “gene” refers to a DNA segment that is involved in producing a polypeptide and includes regions preceding and following the coding regions as well as intervening sequences (introns) between individual coding segments (exons).

The terms “polynucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. Polynucleotides may be single- or multi-stranded (e.g., single-stranded, double-stranded, and triple-helical) and contain deoxyribonucleotides, ribonucleotides, and/or analogs or modified forms of deoxyribonucleotides or ribonucleotides, including modified nucleotides or bases or their analogs. Because the genetic code is degenerate, more than one codon may be used to encode a particular amino acid, and the present invention encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, and the like). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need and circular portions. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, cDNA, recombinant polynucleotides, branched polynucleotides, plasmids, vectors, isolated DNA of any sequence, isolated RNA of any sequence, nucleic acid probes, adapters, and primers. A polynucleotide may include modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component, tag, reactive moiety, or binding partner. Polynucleotide sequences, when provided, are listed in the 5′ to 3′ direction, unless stated otherwise.

As used herein, “polypeptide” refers to a composition comprised of amino acids and recognized as a protein by those of skill in the art. The conventional one-letter or three-letter code for amino acid residues is used herein. The terms “polypeptide” and “protein” are used interchangeably herein to refer to polymers of amino acids of any length. The polymer may be linear or branched, it may include modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, synthetic amino acids and the like), as well as other modifications known in the art.

Computer Systems

The present invention is described partly in terms of functional components and various processing steps. Such functional components and processing steps may be realized by any number of components, operations and techniques configured to perform the specified functions and achieve the various results. For example, the present invention may employ various biological samples, biomarkers, elements, materials, computers, data sources, storage systems and media, information gathering techniques and processes, data processing criteria, statistical analyses, regression analyses and the like, which may carry out a variety of functions. In addition, although the invention is described in relation to genetic analysis, the present invention may be practiced in conjunction with any number of applications, environments and data analyses; the systems described herein are merely exemplary applications for the invention.

Methods for genetic analysis according to various aspects of the present invention may be implemented in any suitable manner, for example using a computer program operating on the computer system. An exemplary genetic analysis system, according to various aspects of the present invention, may be implemented in conjunction with a computer system, for example a conventional computer system comprising a processor and a random access memory, such as a remotely-accessible application server, network server, personal computer or workstation. The computer system also suitably includes additional memory devices or information storage systems, such as a mass storage system and a user interface, for example a conventional monitor, keyboard and tracking device. The computer system may, however, comprise any suitable computer system and associated equipment and may be configured in any suitable manner. In one embodiment, the computer system comprises a stand-alone system. In another embodiment, the computer system is part of a network of computers including a server and a database.

The software required for receiving, processing, and analyzing genetic information may be implemented in a single device or implemented in a plurality of devices. The software may be accessible via a network such that storage and processing of information takes place remotely with respect to users. The genetic analysis system according to various aspects of the present invention and its various elements provide functions and operations to facilitate genetic analysis, such as data gathering, processing and/or analysis. The present genetic analysis system maintains information relating to samples and facilitates analysis, For example, in the present embodiment, the computer system executes the computer program, which may receive, store, search, analyze, and report information relating to the genome. The computer program may comprise multiple modules performing various functions or operations, such as a processing module for processing raw data and generating supplemental data and an analysis module for analyzing raw data and supplemental data to perform genetic analysis.

The procedures performed by the genetic analysis system may comprise any suitable processes to facilitate genetic analysis. In one embodiment, the genetic analysis system is configured to identify gene variants.

The genetic analysis system may also provide various additional modules and/or individual functions. For example, the genetic analysis system may also include a reporting function, for example to provide information relating to the processing and analysis functions. The genetic analysis system may also provide various administrative and management functions, such as controlling access and performing other administrative functions.

The following example is provided to further illustrate the advantages and features of the present invention, but it is not intended to limit the scope of the invention. While this example is typical of those that might be used, other procedures, methodologies, or techniques known to those skilled in the art may alternatively be used.

### Example I

### MASSIVELY PARALLEL MATHOD TO ASSAY SEIZURE GENES IN C. Elegans

The following methodology will be utilized to assay seizure genes using a worm model.

The methodology will include the following:

a) identify a human gene that is orthologous (human ortholog) to a worm gene that, when dysregulated in the worm, causes a defect in chemotaxis (such as CACNB4);
b) humanize the worm, by introducing the human ortholog cDNA into the worm, and observing that it restores chemotaxis (aka gene rescue);
c) generate gene variant oligonucleotides of ortholog gene variants having all possible amino acid substitutions;
d) transfect a C. elegans population with the gene variant oligos;
e) high throughput sequence a subset of these worms to measure the relative proportions of each variant;
f) conduct a chemotaxis assay; and
g) high-throughput sequence the worms that responded to the stimulus in the chemotaxis assay (either by moving toward or away from it) and those that did not to measure the relative abundance of each variant in each group.

Testing for drug efficacy may be conducted in a similar fashion except step (f) is conducted in the presence of a drug that is expected to alter function of the targeted protein.

### Example II

### Assay of CACNB4

The methodology set forth in Example I was performed using CACNB4 as the human ortholog.

For the chemotaxis assay, worms were placed on an agar plate or similar. Attractant was placed at some distance from the worms. After some time (typically 30-60 minutes) the number of worms within a certain distance of the attractant (typically 5 mm) were counted and divided by the total count of worms. Alternatively, this was also scored by determining how many worms move significantly (e.g. 5 mm) from their initial starting point.

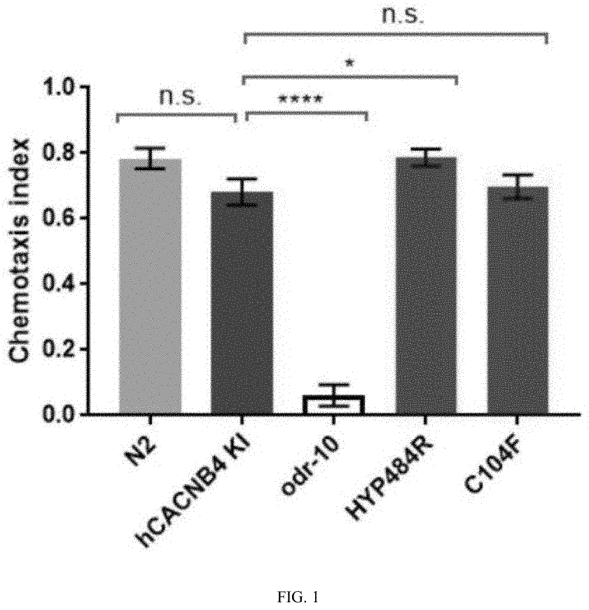

FIG. 1 shows relative chemotaxis of worms with humanized CACNB4 having particular mutations. N2—wild type worm. hCACNB4, wild type work with CACNB4 ortholog replaced by human CACNB4. Odr-10 odorant receptor 10 null worm (negative control). HYPR484R, variant of uncertain significance. C104F, known pathogenic mutation.

Although the invention has been described with reference to the above examples, it will be understood that modifications and variations are encompassed within the spirit and scope of the invention. Accordingly, the invention is limited only by the following claims.

What is claimed is:

1. A method comprising:
a) humanizing a worm by integrating a human ortholog into the worm genome;
b) transfecting a worm population with a library of oligonucleotides wherein the library of oligonucleotides contain genetic variants of the ortholog;
c) detecting the relative proportions of each genetic variant in the worm population;
d) conducting a chemotaxis assay using worms of the worm population; and
e) detecting worms that respond to a stimulus in the chemotaxis assay and worms that fail to respond to the stimulus and measuring the relative proportion of each ortholog variant in each group of worms.

2. The method of claim 1, wherein the library comprises oligonucleotides having at least 90% or more of all possible amino acid substitutions, deletions and/or mutations.

3. The method of claim 1, wherein the library comprises oligonucleotides having all possible amino acid substitutions, deletions and/or mutations.

4. The method of claim 1, wherein the chemotaxis assay is conducted in the presence of a test compound which alters function of the ortholog, wherein the altered function is a change in chemotaxis activity.

5. The method of claim 1, wherein the worm population is Caenorhabditis elegans.

6. The method of claim 1, wherein (c) and (e) comprise high throughput sequencing.

7. The method of claim 1, wherein the ortholog causes a defect in chemotaxis when altered in the worm.

8. The method of claim 7, wherein the ortholog is CACNB4 or STXBP1.

9. Use of the method of any of claims 1 to 8 to identify genetic variants of a gene.

10. Use of the method of any of claims 1 to 8 to classify a genetic variant as pathogenic or non-pathogenic.

11. Use of the method of any of claims 1 to 8 to perform drug screening in a mutation specific manner.

12. A system comprising:
a controller including at least one processor and non-transitory memory, wherein the controller is configured to perform (b), (c) and/or (e) of claim 1.

13. A method for identifying a pathogenic genetic mutation in a gene using a human worm ortholog model comprising:
a) humanizing a worm by integrating a human ortholog into the worm genome;
b) transfecting a worm population with a library of oligonucleotides wherein the library of oligonucleotides contain genetic variants of the ortholog;
c) detecting the relative proportions of each genetic variant in the worm population;
d) conducting a chemotaxis assay using worms of the worm population; and
e) detecting worms that respond to a stimulus in the chemotaxis assay and worms that fail to respond to the stimulus and measuring the relative proportion of each ortholog variant in each group of worms, wherein worms that do not respond to the stimulus have a genetic variant of the ortholog that is pathogenic, thereby identifying a pathogenic genetic mutation.

14. The method of claim 13, wherein the library comprises oligonucleotides having at least 90% or more of all possible amino acid substitutions, deletions and/or mutations.

15. The method of claim 13, wherein the library comprises oligonucleotides having all possible amino acid substitutions, deletions and/or mutations.

16. The method of claim 13, wherein the chemotaxis assay is conducted in the presence of a test compound which alters function of the ortholog, wherein the altered function is a change in chemotaxis activity.

17. The method of claim 13, wherein the worm population is Caenorhabditis elegans.

18. The method of claim 13, wherein (c) and (e) comprise high throughput sequencing.

19. The method of claim 13, wherein the ortholog causes a defect in chemotaxis when altered in the worm.

20. The method of claim 13, wherein the ortholog is CACNB4 or STXBP1.

21. A system comprising:
a controller including at least one processor and non-transitory memory, wherein the controller is configured to perform (b), (c) and/or (e) of claim 13.

22. A method for screening a test agent in a human worm ortholog model comprising:
a) humanizing a worm by integrating a human ortholog into the worm genome;
b) transfecting a worm population with a library of oligonucleotides wherein the library of oligonucleotides contain genetic variants of the ortholog;
c) detecting the relative proportions of each genetic variant in the worm population;
d) conducting a chemotaxis assay using worms of the worm population, wherein the assay is conducted in the presence and absence of a test compound; and
e) determining whether the test compound alters function of the ortholog by detecting an increase or decrease in chemotaxis activity by comparing chemotaxis activity in the presence and absence of the compound.

23. The method of claim 22, wherein the library comprises oligonucleotides having at least 90% or more of all possible amino acid substitutions, deletions and/or mutations.

24. The method of claim 22, wherein the library comprises oligonucleotides having all possible amino acid substitutions, deletions and/or mutations.

25. The method of claim 22, wherein the worm population is Caenorhabditis elegans.

27. The method of claim 22, wherein (c) comprise high throughput sequencing.

28. The method of claim 22, wherein the ortholog causes a defect in chemotaxis when altered in the worm.

29. The method of claim 22, wherein the ortholog is CACNB4 or STXBP1.

30. A system comprising:
a controller including at least one processor and non-transitory memory, wherein the controller is configured to perform (b) and/or (c) of claim 22.
